(19) United States
(12) Patent Application Publication
(10) Pub. No.: US 2024/0050533 A1
(43) Pub. Date: Feb. 15, 2024

(54) ORAL OCTREOTIDE THERAPY IN COMBINATION WITH DIGOXIN OR LISINOPRIL

(71) Applicant: Amryt Endo, Inc., Boston, MA (US)

(72) Inventors: Sam L. TEICHMAN, Lafayette, CA (US); Asi HAVIV, Mazkeret Batya (IL); Jennings Ray DAWKINS, Coats, NC (US); Ruth Engle STEVENS, Long Beach, WA (US)

(21) Appl. No.: 18/335,673

(22) Filed: Jun. 15, 2023

Related U.S. Application Data
(63) Continuation of application No. 17/135,081, filed on Dec. 28, 2020.

(51) Int. Cl. A61K 38/31; A61K 31/401; A61K 9/00; A61K 31/7048

(52) U.S. Cl. CPC A61K 38/31; A61K 31/401; A61K 9/0053; A61K 31/7048

(57) ABSTRACT

This invention relates to methods of co-administering oral octreotide and digoxin or lisinopril to a subject in need thereof.

### FIELD OF THE INVENTION

This invention relates to methods of co-administering oral octreotide and digoxin or lisinopril to a subject in need thereof.

### BACKGROUND

Although octreotide has been known as an injectable medicament for many years, it is only very recently that oral octreotide has been developed. Utilizing its proprietary formulation designated TPE® (transient permeability enhancer), Chiasma, Inc developed a new formulation of octreotide acetate for oral delivery and entered the market under the brand name MYCAPSSA® in June 2020. The TPE formulation facilitates intestinal absorbance of drug molecules with limited intestinal bioavailability; the formulation protects the drug molecule from inactivation by the hostile gastrointestinal (GI) environment and at the same time acts on the GI wall to induce a transient and reversible opening of the paracellular route allowing permeation of the drug molecules through the tight junctions; see Tuvia (2014) Pharm Res 31:2010-2021. These two attributes ensure that the drug molecule reaches the bloodstream effectively in its active form. TPE is a combination of excipients assembled in a process leading to an oily suspension of hydrophilic particles containing medium-chain fatty acid salts and the active pharmaceutical ingredient (herein octreotide) suspended in a lipophilic medium.

It was contemplated that the formulation of octreotide acetate for oral delivery might affect the bioavailability of other drugs administered to patients in need of oral octreotide therapy. Potential drug-drug interactions of the oral octreotide formulation with such drugs are herein evaluated.

### SUMMARY

The inventors of the present invention have discovered that the rate of digoxin absorption (bioavailability of digoxin) is decreased when administered to a subject who is concomitantly being administered oral octreotide. The inventors of the present invention have also discovered that the absorption of lisinopril (bioavailability of lisinopril) is increased when administered to a subject who is concomitantly being administered oral octreotide.

The inventors of the present invention have invented a method of treating a subject in need of oral octreotide and digoxin, the method comprising:
i) co-administering oral octreotide and digoxin to the subject;
ii) assessing the clinical response of the subject to digoxin; and
iii) if the clinical response of digoxin is altered when the subject is co-administered oral octreotide relative to the clinical response of digoxin in the absence of oral octreotide, then adjusting the dose of digoxin administered to the subject.

The inventors of the present invention have discovered a method of treating a subject in need of oral octreotide and digoxin, the method comprising:
i) administering to the subject oral octreotide, during concomitant administration with digoxin;
ii) assessing the clinical response of the subject to digoxin; and
iii) adjusting the dose of digoxin administered to the subject depending on the assessment of the clinical response of the subject to digoxin.

The inventors of the present invention have discovered a method of treating a subject in need of oral octreotide and lisinopril, the method comprising:
i) co-administering oral octreotide and lisinopril to the subject;
ii) monitoring the blood pressure of the subject; and
iii) if the blood pressure of the subject is reduced when the subject is co-administered oral octreotide relative to the blood pressure of the subject in the absence of oral octreotide, then adjusting the dose of lisinopril administered to the subject.

Moreover, it is to be understood that both the foregoing information and the following detailed description are merely illustrative examples of various aspects and embodiments, and are intended to provide an overview or framework for understanding the nature and character of the claimed aspects and embodiments. The accompanying drawings are included to provide illustration and a further understanding of the various aspects and embodiments and are incorporated in and constitute a part of this specification. The drawings, together with the remainder of the specification, explain principles and operations of the described and claimed aspects and embodiments.

Throughout this application, various publications, including United States patents, are referenced by author and year and patents and applications by number. The disclosures of these publications and patents and patent applications in their entireties are hereby incorporated by reference into this application in order to more fully describe the state of the art to which this invention pertains.

### BRIEF DESCRIPTION OF THE DRAWINGS

Various aspects of at least one embodiment are discussed below with reference to the accompanying Figures. The Figures are provided for the purposes of illustration and explanation and are not intended as a definition of the limits of the invention. The Figures are as referenced in the accompanying Example.

In the Figures:

FIG. 1 presents pharmacokinetic data of oral octreotide after administration with 240 mL and 75 mL of water,

FIG. 2 presents pharmacokinetic data of digoxin with placebo and with oral octreotide; and

FIG. 3 presents pharmacokinetic data of lisinopril with placebo and with oral octreotide.

### DETAILED DESCRIPTION OF THE INVENTION

Octreotide is a synthetic octapeptide analog of human somatostatin, a naturally occurring tetradecapeptide. Octreotide has been approved for chronic use in acromegaly patients as a life-long treatment by the FDA since 1988 as Sandostatin®, produced by Novartis. Long acting depot octreotide, octreotide LAR (Sandostatin LAR) was approved by the FDA in 1998 for the treatment of acromegaly and diarrhea associated with carcinoid syndrome and with vasoactive intestinal peptide secreting adenomas. Both forms of octreotide are administered parenterally; in the past octreotide acetate when given orally had insufficient bioavailability.

Identifying the need for a less difficult (less cumbersome) alternative to the currently available octreotide injections, Chiasma, Inc developed a new formulation of octreotide acetate for oral delivery utilizing its proprietary formulation designated Transient Permeability Enhancer (TPE®), and the product is called MYCAPSSA®. See for example co-assigned U.S. Pat. Nos. 8,535,695 and 10,695,397.

MYCAPSSA is a delayed-release capsule which enables the oral delivery of octreotide acetate. MYCAPSSA was approved by the FDA on 26 Jun. 2020 for long-term maintenance treatment in acromegaly patients who have responded to and tolerated treatment with octreotide or lanreotide.

MYCAPSSA (octreotide) delayed-release capsule is a combination of octreotide acetate and excipients collectively called Transient Permeability Enhancer (TPE®). TPE is a proprietary excipient mixture that permits oral administration, comprised of the following: polyvinylpyrrolidone (PVP-12), sodium caprylate (octanoate), magnesium chloride, polysorbate 80, glyceryl monocaprylate and glyceryl tricaprylate.

Without being bound by theory, the TPE improves the oral bioavailability of poorly absorbed drugs such as octreotide by increasing the permeability of the intestine. The mode of action of TPE is thought to involve a transient opening of the tight junctions between epithelial cells lining the intestine; see Tuvia (2014) Pharm Res 31:2010-2021.

Oral octreotide acetate (herein also termed oral octreotide; the terms are used interchangeably herein) is supplied in an enteric-coated capsule filled with an oily suspension of octreotide acetate formulated with TPE. The enteric coating allows the intact capsule to pass through the stomach and disintegrate when it reaches the higher pH of the small intestine to discharge oral octreotide acetate suspension.

The TPE facilitates intestinal absorbance of drug molecules with limited intestinal bioavailability. The TPE formulation protects the drug molecule from inactivation by the hostile gastrointestinal (GI) environment and at the same time acts on the GI wall to induce a transient and reversible opening of the paracellular route allowing permeation of the drug molecules through the tight junctions. These two attributes ensure that when delivered in TPE formulation, the drug reaches the bloodstream effectively in its native active form. TPE is a combination of excipients assembled in a process leading to an oily suspension of hydrophilic particles containing medium-chain fatty acid salts and the active pharmaceutical ingredient (API) suspended in a lipophilic medium.

The permeation enhancement effect of the TPE formulation in oral octreotide acetate might affect the bioavailability of other orally administered drugs commonly used by acromegaly patients. Therefore, the potential for drug-drug interaction (DDI) of oral octreotide acetate with oral representative probe drugs was evaluated in this study. Probe drugs with the following specific characterizations were included: lisinopril was selected as a drug with low permeability, and digoxin was selected as a drug with a narrow therapeutic window. These two probe dugs were evaluated in this study.

### Digoxin:

Digoxin is a drug that affects the myocardium.

The inventors of the present invention have invented a method of treating a subject in need of oral octreotide and digoxin, the method comprising:
i) co-administering digoxin and oral octreotide to the subject;
ii) assessing the clinical response of the subject to digoxin; and
iii) if the clinical response of digoxin is altered when the subject is co-administered oral octreotide relative to the clinical response of digoxin in the absence of oral octreotide, then adjusting the dose of digoxin administered to the subject.

In another aspect, provided herein is a method of treating a subject in need of oral octreotide and digoxin, the method comprising:
i) co-administering digoxin and a therapeutically effective amount of oral octreotide to the subject;
ii) assessing the clinical response of the subject to digoxin; and
iii) if the clinical response of digoxin is altered when the subject is co-administered oral octreotide relative to the clinical response of digoxin in the absence of oral octreotide, then adjusting the dose of digoxin administered to the subject.

In an embodiment, the clinical response of digoxin is the level of digoxin, rate of digoxin absorption, bioavailability of digoxin, or peak exposure of digoxin in the subject or steady state level of serum digoxin in the subject.

In an embodiment of the invention the method further comprises assessing the level of digoxin in the subject (e.g., the level of digoxin in a serum sample of the subject).

In an embodiment of the invention the level of digoxin is decreased when the subject is co-administered oral octreotide relative to a reference standard (e.g., level of digoxin in a serum sample of the subject in the absence of oral octreotide (e.g., level of digoxin in a serum sample prior to administration of oral octreotide)).

In an embodiment of the invention the method further comprises assessing the rate of digoxin absorption in the subject.

In an embodiment of the invention the rate of digoxin absorption is decreased when the subject is co-administered oral octreotide relative to a reference standard (e.g., rate of digoxin absorption in the subject in the absence of oral octreotide).

In an embodiment of the invention the rate of digoxin absorption is decreased when the subject is co-administered oral octreotide relative to the rate of digoxin absorption in the absence of oral octreotide.

In an embodiment of the invention the method further comprises assessing the bioavailability of digoxin in the subject.

In an embodiment of the invention the bioavailability of digoxin is decreased when the subject is co-administered oral octreotide relative to a reference standard (e.g., bioavailability of digoxin in the subject in the absence of oral octreotide).

In an embodiment of the invention the rate of bioavailability of digoxin is decreased when the subject is co-administered oral octreotide relative to the bioavailability of digoxin in the absence of oral octreotide.

In an embodiment of the invention the method further comprises assessing the peak exposure (Cₘₐₓ) of digoxin in the subject. In some embodiments, Cₘₐₓ of digoxin is decreased when the subject is co-administered oral octreotide relative to a reference standard (e.g., peak exposure Cₘₐₓ of digoxin in the subject in the absence of oral octreotide).

In some embodiments the digoxin exposure is measured clinically with a blood test.

In some embodiments of the invention, the reference standard is a patient sample (e.g., patient sample acquired prior to administering oral octreotide to the subject or patient sample in the absence of oral octreotide). In some embodiments, the reference standard may be a serum or blood sample of the subject.

In another embodiment of the invention, the method further comprises monitoring serum digoxin levels in the subject when concomitant therapy with oral octreotide is initiated.

Adjusting the dose of digoxin (e.g., LANOXIN®) administered to the subject may be performed as described in the package insert for LANOXIN (Prescribing Information, GlaxoSmithKline).

As used herein “assessment of the clinical response of the subject to digoxin” is as described in the package label (prescribing information) for LANOXIN® (digoxin) tablets (GlaxoSmithKline) “In general, the dose of digoxin used should be determined on clinical grounds. However, measurement of serum digoxin concentrations can be helpful to the clinician in determining the adequacy of digoxin therapy and in assigning certain probabilities to the likelihood of digoxin intoxication.” Also: “Maintenance Dosing: The doses of digoxin used in controlled trials in patients with heart failure have ranged from 125 to 500 mcg (0.125 to 0.5 mg) once daily. In these studies, the digoxin dose has been generally titrated according to the patient's age, lean body weight, and renal function. Therapy is generally initiated at a dose of 250 mcg (0.25 mg) once daily in patients under 70 with good renal function, at a dose of 125 mcg (0.125 mg) once daily in patients over 70 or with impaired renal function, and at a dose of 62.5 mcg (0.0625 mg) in patients with marked renal impairment. Doses may be increased every 2 weeks according to clinical response.

Thus careful assessment of clinical response should be performed when digoxin is concomitantly administered with MYCAPSSA, including monitoring of digoxin blood levels; it is likely that the individual maintenance dose of a subject on digoxin therapy will have to be adjusted (e.g., titrated up) on starting oral octreotide therapy since the rate of digoxin absorption is decreased when administered concomitantly with oral octreotide therapy. It would be appropriate to recommend monitoring of digoxin levels and efficacy when concomitant therapy with oral octreotide acetate is initiated.

Further information on dose selection for digoxin may be found in Collier et al (1978) J Pharm 1(1):3-14.

The “clinical grounds” for the assessment of the clinical response of the subject to digoxin may be carried out by physical evaluation, including measurement of heart rate and/or by obtaining ECG results.

As used herein, a subject “in need of digoxin therapy” is a subject who would benefit from administration of digoxin. The subject may be suffering from any disease or condition wherein digoxin therapy may be useful in ameliorating symptoms. Such diseases or conditions include heart failure, atrial fibrillation and pulmonary arterial hypertension (PAH). See Chen et al (2015) U.S. Pharm. 40(2):44-48.

### Lisinopril:

Lisinopril is a drug indicated for treatment of high blood pressure (hypertension), adjunct therapy for heart failure and treatment of acute myocardial infarction.

One embodiment of the invention relates to a method of treating a subject in need of oral octreotide and lisinopril, the method comprising:
i) co-administering oral octreotide and lisinopril to the subject;
ii) monitoring the blood pressure of the subject; and
iii) if the blood pressure of the subject is reduced when the subject is co-administered oral octreotide relative to the blood pressure of the subject in the absence of oral octreotide, then adjusting the dose of lisinopril administered to the subject.

One embodiment of the invention relates to a method of treating a subject in need of oral octreotide and lisinopril, the method comprising:
i) co-administering lisinopril and a therapeutically effective amount of octreotide to the subject;
ii) monitoring the blood pressure of the subject; and
iii) if the blood pressure of the subject is reduced when the subject is co-administered oral octreotide relative to the blood pressure of the subject in the absence of oral octreotide, then adjusting the dose of lisinopril administered to the subject.

In one embodiment of the invention the blood pressure of the subject is reduced relative to a reference standard (e.g., blood pressure of the subject in the absence of oral octreotide, blood pressure of the subject prior to administering oral octreotide to the subject, or wherein the reference standard is normal blood pressure which is less than 120/80 (120 mm Hg (systolic)/80 mm Hg (diastolic))).

In another embodiment of the invention the method further comprises assessing the bioavailability of lisinopril.

In an embodiment of the invention the bioavailability of lisinopril may be increased when the subject is co-administered oral octreotide relative to a reference standard (e.g., bioavailability of lisinopril in the absence of oral octreotide or bioavailability of lisinopril prior to administering oral octreotide to the subject or wherein the reference standard is normal blood pressure which is less than 120/80 (120 mm Hg (systolic)/80 mm Hg (diastolic))).

Adjusting the dose of lisinopril (e.g., Zestril®) administered to the subject may be performed as described in the package insert for Zestril (Prescribing Information, AstraZeneca) as follows:
“Hypertension: Initial adult dose is 10 mg once daily. Titrate up to 40 mg daily based on blood pressure response. Initiate patients on diuretics at 5 mg once daily.
Heart failure: Initiate with 5 mg once daily. Increase dose as tolerated to 40 mg daily
Acute myocardial infarction (MI): Give 5 mg within 24 hours of MI followed by 5 mg after 24 hours then 10 mg once daily.”

Further information on dose selection for lisinopril may be found in Beermann et al (1989) Biopharm Drug Dispos 10(4):397-409. Further information on blood pressure can be found in The Seventh Report of the Joint National Committee on Prevention, Detection, Evaluation and Treatment of High Blood Pressure, NIH Publication No. 03-5233, December 2003.

Titration up of lisinopril is described above when prescribing lisinopril for the first time. Titration down can be performed if needed when a patient being administered lisinopril commences octreotide therapy, in order to avoid excess lowering of blood pressure in the subject in need of oral octreotide therapy.

As used herein, a subject “in need of lisinopril therapy” is a subject who would benefit from administration of lisinopril. The subject may be suffering from any disease or condition wherein lisinopril therapy may be useful in ameliorating symptoms. Such diseases or conditions include high blood pressure (hypertension), adjunct therapy for heart failure and treatment of acute myocardial infarction. See Zestril (lisinopril) package insert (AstraZeneca). See also Beermann et al. (1989) Biopharm Drug Dispos 10(4): 397-409.

### Oral Octreotide:

As used herein, a subject “in need of oral octreotide” is a subject who would benefit from administration of oral octreotide. The subject may be suffering from any disease or condition for which oral octreotide therapy may be useful in ameliorating symptoms. Such diseases or conditions include acromegaly, abnormal GI motility, carcinoid syndrome, flushing associated with carcinoid syndrome or flushing associated with a metastatic carcinoid tumor, diarrhea associated with carcinoid syndrome in particular severe diarrhea, intractable or severe diarrhea, portal hypertension, a neuroendocrine tumor, a vasoactive intestinal peptide secreting adenoma, diarrhea associated with a vasoactive intestinal peptide secreting adenoma in particular severe diarrhea, flushing associated with a vasoactive intestinal peptide secreting adenoma, gastroparesis, diarrhea, pancreatic leak or pancreatic pseudo-cysts or portal hypertension polycystic disease e.g., polycystic kidney disease or polycystic liver disease or PCOS or hypotension especially neurogenic orthostatic hypotension and postprandial hypotension. In a certain embodiment of the invention the subject has acromegaly; in another embodiment of the invention the subject has severe diarrhea or flushing episodes associated with metastatic carcinoid tumor.

The subject may need oral octreotide to prevent variceal bleeding (e.g., bleeding varices such as bleeding esophageal varices or bleeding gastric varices).

Oral octreotide is indicated for symptomatic treatment of patients with metastatic carcinoid tumors where it suppresses or inhibits the severe diarrhea and flushing episodes associated with the disease.

The term “therapeutically effective amount,” as used herein, refers to an amount of a compound sufficient to treat, ameliorate, or prevent the identified disease or condition, or to exhibit a detectable therapeutic, prophylactic, or inhibitory effect. The effect can be detected by, for example, an improvement in clinical condition, or reduction in symptoms. The precise effective amount for a subject will depend upon the subject's body weight, size, and health; the nature and extent of the condition; and the therapeutic or combination of therapeutics selected for administration.

The terms “concomitant,” “co-administration” and “co-administering” and their grammatical equivalents, as used herein, encompass administration of two or more agents to the subject so that both agents and/or their metabolites are present in the subject at the same or substantially the same time. The second agent may have been administered to the patient subsequently, simultaneously, or prior to the administration of the first agent.

### Package Insert Instructions

In one aspect of the invention, a package or kit is provided comprising capsules of oral octreotide, and a package insert, package label, instructions or other labeling including any one, two, three or more of the following information or recommendations regarding digoxin:
(i) advising that, when a subject is administered oral octreotide, if digoxin is co-administered then the clinical response of the subject to digoxin must be carefully assessed;
(ii) careful assessment of clinical response should be performed when digoxin is concomitantly administered with MYCAPSSA;
(iii) monitoring of digoxin levels and efficacy should be performed when concomitant therapy with oral octreotide acetate is initiated:
and (iv) if the clinical response of digoxin is altered when the subject is co-administered oral octreotide relative to clinical response of digoxin in the absence of oral octreotide, then the dose of digoxin administered to the subject should be adjusted.

In another aspect of the invention, a package or kit is provided comprising capsules of oral octreotide, and a package insert, package label, instructions or other labeling including any one, two, three or more of the following information or recommendations regarding lisinopril:
(i) advising that when a subject is co-administered oral octreotide and lisinopril then the blood pressure of the subject should be monitored and if the blood pressure of the subject is reduced when the subject is co-administered oral octreotide relative to the blood pressure of the subject in the absence of oral octreotide, then the dose of lisinopril administered to the subject should be adjusted;
(ii) monitor patient's blood pressure and adjust the dosage of lisinopril if needed;
(iii) the recommended course of action is the consideration of a dose reduction at the initiation of therapy with oral octreotide acetate. The degree of adjustment will be dictated by the clinical effects;
(iv) monitoring of digoxin levels and efficacy when concomitant therapy with oral octreotide acetate is initiated.

In other embodiments, the information or recommendation may include that administration of oral octreotide to a patient who is being treated with digoxin can alter the therapeutic effect of the digoxin (e.g., can reduce peak exposure Cₘₐₓ of the digoxin). In other embodiments, the information or recommendation may include that co-administration of oral octreotide with digoxin can alter the bioavailability of the digoxin (e.g., can reduce exposure to digoxin). In an embodiment, it would be appropriate to recommend monitoring of digoxin levels and efficacy when concomitant therapy with oral octreotide acetate is initiated. It is also appropriate to recommend that careful assessment of clinical response should be performed when digoxin is concomitantly administered with MYCAPSSA.

In a related aspect, the invention provides a method of preparing or packaging an oral octreotide medicament comprising packaging capsules of oral octreotide together with a package insert or package label or instructions including any one or two or more of the foregoing information or recommendations.

The package insert, package label, instructions or other labeling may further comprise directions for treating a patient suffering from acromegaly or suffering from severe diarrhea or flushing episodes associated with metastatic carcinoid tumor by administering oral octreotide, e.g., at a dosage of between 20 to 200 mg per day (i.e., daily). Dosages envisaged are 20, 30, 40, 50, 60, 70, 80, 90, 100, 110, 120, 130, 140, 150, 160, 170, 180, 190 or 200 mg per day. These dosages may be divided and administered once per day or twice per day or three times per day.

In some embodiments of the invention the oral octreotide is administered in capsules. The capsule may contain 5-50 mg octreotide in particular 5-30 mg octreotide. The amount of octreotide which may be in each capsule are 5, 6, 7, 8, 9, 10, 11, 12, 13, 14, 15, 16, 17, 18, 19, 20, 21, 22, 23, 24, 25, 30, 35, 40, 45 or 50 mg per capsule. In a particular embodiment there is 20 mg octreotide per capsule. In another particular embodiment there is 10 mg per octreotide capsule. In other embodiments there is 7-15 mg octreotide per capsule, e.g., 7, 8, 9, 10 11, 12, 13, 14 or 15 mg per capsule.

The octreotide capsule described herein (e.g., MYCAPSSA) is an oral product indicated for long-term maintenance therapy in acromegaly patients; in certain embodiments the patients are those in whom prior treatment with somatostatin analogs (by injection) has been shown to be effective and tolerated. The goal of treatment in acromegaly is to control GH and IGF-1 levels and to lower the GH and IGF-1 levels to as close to normal as possible.

The oral octreotide product should preferably be administered with a glass of water on an empty stomach (i.e., at least 1 hour prior to a meal or at least 2 hours after a meal).

Patients currently receiving somatostatin analog therapy by injection can be switched to octreotide capsules with an initial dose of 40 mg octreotide (e.g., 20 mg BID) given orally. Blood levels of IGF-1 and clinical symptoms should be monitored. If IGF-1 is normal and clinical symptoms are controlled or response level (biochemical and symptomatic response) is maintained, maintain oral octreotide dosage at 40 mg daily (e.g., 20 mg BID). Dosage may be adjusted to 60 mg of oral octreotide daily (e.g., 40 mg morning+20 mg evening) if IGF-1 levels are increased, as determined by the treating physician, or in case of symptomatic exacerbation. Monitoring is continued, while applying the above algorithm for maintaining or increasing the dose up to 80 mg of oral octreotide daily (e.g., 40 mg BID). The administering throughout occurs at least 2 hours after a meal, or at least 1 hour before a meal.

Additionally, if a capsule containing about 30 mg octreotide is administered, then the above algorithm is used to adjust the dose from 60 mg daily to 90 mg daily and a maximum of 120 mg daily; wherein the administering occurs at least 2 hours after a meal, or at least 1 hour before a meal. In another embodiment, if a capsule containing about 30 mg octreotide is administered, then the above algorithm is used to adjust the dose from 30 mg daily (only one capsule taken) to 60 mg daily to 90 mg daily and a maximum of 120 mg daily; wherein the administering occurs at least 2 hours after a meal, or at least 1 hour before a meal.

Furthermore, if a capsule containing less than 20 mg octreotide is administered e.g., 10 mg, then the above algorithm is adjusted concomitantly. For example, in an embodiment of the invention, if a capsule containing about 10 mg octreotide is administered, then the above algorithm is used to adjust the dose from 20 mg daily to 30 mg daily and a maximum of 60 mg daily as needed; wherein the administering occurs at least 2 hours after a meal, or at least 1 hour before a meal.

The invention may be used in the treatment of naïve patients or patients already treated with parenteral injections (such as parenteral injections of octreotide).

Patients who are not adequately controlled following dose titration can return to therapy by injections at any time. Proton pump inhibitors (PPIs), H2-receptor antagonists, and antacids may lead to a higher dosing requirement of oral octreotide to achieve therapeutic levels.

The invention may be used in the treatment of acromegaly in a subject, the method comprising orally administering to the subject at least once daily at least one dosage form comprising an oily suspension comprising octreotide, wherein the octreotide in each dosage form is from about 5 mg to about 35 mg (e.g., 5, 10, 15, 20, 25, 30 or 35 mg), and wherein the administering occurs at least 1 hour before a meal or at least 2 hours after a meal, to thereby treat the subject. Another embodiment of the invention is a method of treating acromegaly in a subject, the method comprising orally administering to the subject at least once daily at least one dosage form comprising an oily suspension comprising octreotide, wherein the octreotide in each dosage form is from about 18 mg to about 22 mg, and wherein the administering occurs at least 1 hour before a meal or at least 2 hours after a meal to thereby treat the subject.

A dosage form is essentially a pharmaceutical product in the form in which it is marketed for use, typically involving a mixture of active drug components and nondrug components (excipients), along with other non-reusable material that may not be considered either ingredient or packaging (such as a capsule shell, for example).

The oily suspension as used herein comprises an admixture of a hydrophobic medium (lipophilic fraction) and a solid form (hydrophilic fraction) wherein the solid form comprises a octreotide and at least one salt of a medium chain fatty acid, and wherein the medium chain fatty acid salt is present in the composition at an amount of 10% or more by weight such as 11%-15%, or 11%, 12%, 13%, 14%, 15% or more by weight.

Oral formulations of octreotide, comprising the oily suspension incorporated in a capsule, have been described and claimed, for example in co-assigned U.S. Pat. No. 8,329,198 which is hereby incorporated by reference.

Certain embodiments of the invention include a capsule containing a composition comprising a suspension which comprises an admixture of a hydrophobic oily medium and a solid form wherein the solid form comprises a therapeutically effective amount of octreotide, at least one salt of a medium chain fatty acid and polyvinylpyrrolidone (PVP), wherein the polyvinylpyrrolidone is present in the composition at an amount of 3% or more by weight, and wherein the at least one salt of a medium chain fatty acid is present in the composition at an amount of at least 10% by weight.

Certain embodiments of the invention include a capsule containing a composition comprising a suspension which comprises an admixture of a hydrophobic oily medium and a solid form wherein the solid form comprises a therapeutically effective amount of octreotide, at least one salt of a medium chain fatty acid and polyvinylpyrrolidone (PVP), wherein the polyvinylpyrrolidone is present in the composition at an amount of 3% or more by weight, and wherein the at least one salt of a medium chain fatty acid is present in the composition at an amount of at least 12% by weight.

In further embodiments the polyvinylpyrrolidone is present in the composition at an amount of about 5% to about 15% by weight and/or the polyvinylpyrrolidone has a molecular weight of about 3000; and/or the medium chain fatty acid salt has a chain length from about 6 to about 14 carbon atoms and/or the medium chain fatty acid salt is sodium hexanoate, sodium heptanoate, sodium octanoate, sodium nonanoate, sodium decanoate, sodium undecanoate, sodium dodecanoate, sodium tridecanoate or sodium tetradecanoate, or a corresponding potassium or lithium or ammonium salt or a combination thereof. In a particular embodiment of the invention the medium chain fatty acid salt is sodium octanoate and/or the medium chain fatty acid salt is present in the capsule at an amount of 12% to 18% by weight.

In further embodiments the hydrophobic oily medium within the capsule comprises a mineral oil, a paraffin, a fatty acid a monoglyceride, a diglyceride, a triglyceride, an ether or an ester, or a combination thereof; in a particular embodiment the hydrophobic oily medium comprises glyceryl tricaprylate.

In further embodiments the medium chain fatty acid salt is a lithium, potassium or ammonium salt or is octanoic acid.

In a particular embodiment, the composition within the capsule further comprises a surfactant.

In a particular embodiment of the invention the composition within the capsule comprises a therapeutically effective amount of octreotide and about 12-21% of sodium octanoate, about 5-10% of polyvinylpyrrolidone with a molecular weight of about 3000, about 20-80% of glyceryl tricaprylate, about 0-50% castor oil, about 3-10% surfactant and about 1% water.

In a another embodiment of the invention the composition within the capsule comprises a therapeutically effective amount of octreotide and about 12-21% of sodium octanoate, about 5-10% of polyvinylpyrrolidone with a molecular weight of about 3000, about 20-80% of glyceryl tricaprylate and about 3-10% surfactant.

In another embodiment of the invention the encapsulated composition (composition within the capsule) comprises a therapeutically effective amount of octreotide wherein the octreotide is present at an amount of less than 33%.

In a particular embodiment of the invention the encapsulated composition comprises about 15% of sodium octanoate, about 10% of polyvinylpyrrolidone with a molecular weight of about 3000, about 30-70% glyceryl tricaprylate and about 6% of surfactant e.g., glyceryl monocaprylate and/or polyoxyethylene sorbitan monooleate. In a particular embodiment of the invention the solid form within the encapsulated composition comprises a particle or a plurality of particles.

In another embodiment of the invention the solid form within the encapsulated composition comprises a stabilizer. In particular embodiments of the invention the capsule is enteric coated. In particular embodiments of the invention the octreotide is present in the encapsulated composition at an amount of less than 25%, less than 10%, less than 1%.

In a particular embodiment of the method of the invention the oily suspension is formulated into a capsule, which may be enterically coated. In another embodiment of the method of the invention the capsule consists of an oily suspension. In another embodiment of the method of the invention the subject is dosed every 8-16 hours (e.g., every 12 hours). In another embodiment of the method of the invention one administration takes place at least 6, 8, 10 or 12 hours before a second administration. In a preferred embodiment the subject is a human.

For clarity, the twice daily administration comprises a first administration and a second administration. In a further embodiment a first administration includes one or two dosage forms and a second administration includes one or two dosage forms, and more particularly the first administration includes one dosage form and the second administration includes one dosage form, or the first administration includes two dosage forms and the second administration includes one dosage form, or the first administration includes two dosage forms and the second administration includes two dosage forms. In embodiments of the invention the first administration is in the morning (normally Sam to noon) and the second administration is in the evening (normally 5 pm to midnight). All the administering occurs at least 1 hour before a meal or at least 2 hours after a meal.

Particular embodiments of the invention are as follows: one dosage form is administered twice daily; two dosage forms are administered once a day and one dosage form is administered once a day; and two dosage forms are administered twice daily. Other embodiments of the invention are as follows: one dosage form is administered once a day; two dosage forms are administered once a day; three or more dosage forms are administered once a day; and two or more dosage forms (e.g., three dosage forms) are administered twice a day. All the administering occurs at least 1 hour before a meal or at least 2 hours after a meal.

In some embodiments of the invention, the administration may be self-administration; in other embodiments of the invention or a caregiver or health professional may administer the dosage form.

In certain embodiments of the invention each dosage form comprises from about 19 to about 21 mg of octreotide and in a particular embodiment of the invention each dosage form comprises 20 mg of octreotide which is about 3% w/w octreotide or 3.3% w/w octreotide. In certain embodiments of the invention the total amount of octreotide administered per day is from about 36 to about 44 mg (e.g., from about 38 to about 42 mg, or 40 mg). In certain embodiments of the invention the total amount of octreotide administered per day is from about 54 to about 66 mg (e.g., from about 57 to about 63 mg, or 60 mg). In certain embodiments of the invention the total amount of octreotide administered per day is from about 72 to about 88 mg (e.g., from about 76 to about 84 mg, or 80 mg). In certain embodiments of the invention the total amount of octreotide administered per day is from about 90 to about 110 mg (e.g., from about 95 to about 105 mg, or 100 mg). All the administering occurs at least 1 hour before a meal or at least 2 hours after a meal.

In certain embodiments of the invention each dosage form comprises from about 27 to about 33 mg of octreotide and in a particular embodiment of the invention each dosage form comprises 30 mg of octreotide which is about 5% w/w octreotide or 4.96% w/w octreotide. This may be administered as one, two, three or four capsules per day, wherein administering occurs at least 1 hour before a meal or at least 2 hours after a meal.

In other embodiments of the invention each dosage form comprises less than 20 mg octreotide and in a particular embodiment of the invention each dosage form comprises about 10 mg. This may be administered as one, two, three or four capsules per day, wherein administering occurs at least 1 hour before a meal or at least 2 hours after a meal.

A kit comprising instructions and the dosage form is also envisaged.

In further embodiments, the method of the invention occurs over a duration of at least 7 months, occurs over a duration of at least 13 months and over a duration of greater than 13 months. In a particular embodiment the method of treatment is for long-term maintenance therapy. Long-term maintenance therapy in a subject suffering from acromegaly continues as long as the subject is suffering from acromegaly and the IGF-1 levels are maintained at equal or less than 1.3 times the upper limit of the age-adjusted normal range (ULN). Thus the duration may be unlimited. In particular embodiments the long-term maintenance therapy may be for at least one, two, three, four or five years and more. In a particular embodiment upon administration of octreotide, an in vivo amount of growth hormone integrated over 2 hours is obtained which is equal or less than 2.5 ng/mL or equal or less than 1.0 ng/mL.

In further embodiments, upon administration of octreotide, an in vivo concentration of IGF-I is obtained of equal or less than 1.3 times the upper limit of the age-adjusted normal range (ULN), or equal or less than 1.0 or 1.1 or 1.2 or 1.4 or 1.5 or 1.6 times the upper limit of the age-adjusted normal range (ULN).

In certain embodiments, an in vivo mean peak plasma concentration upon administration of octreotide of about 3.5+/−0.5 ng/mL is achieved. In certain embodiments an in vivo mean area under the curve upon administration of octreotide is about 15+/−4 h×ng/mL is obtained.

In particular embodiments of the method of the invention the subject has had prior treatment for acromegaly, and the prior treatment for acromegaly was surgical and/or medicinal; in certain embodiments the medicinal treatment was a somatostatin analog (=somatostatin receptor ligand) e.g., injectable octreotide or injectable lanreotide or injectable pasireotide and/or a dopamine agonist e.g., cabergoline and/or a GH receptor antagonist e.g., pegvisomant.

In particular embodiments the prior treatment of the subject with a somatostatin analog has been shown to be effective and tolerated.

In particular embodiments the prior treatment of the subject produced an IGF-1 level in the subject of equal or less than 1.3 times upper limit of normal (ULN), and/or prior treatment of the subject produced 2-hour integrated growth hormone (GH) of less than 2.5 ng/mL or less than 1.0 ng/mL.

Preferably the oral octreotide capsule should be administered on an empty stomach (i.e., at least 1 hour prior to a meal or at least 2 hours after a meal. In particular embodiments of all inventions described herein, a meal comprises 100-1000 calories, or 300-600 calories which may be a high-fat meal or a high calorie meal and may comprise carbohydrates and/or fat and or protein e.g., 100, 200, 300, 400 calories or 500-1000 calories or 700-800 calories.

The invention also contemplates titrating a patient suffering from acromegaly to determine the effective dose of octreotide. Such an embodiment of the invention relates to a method of titrating a patient having acromegaly, the method comprising orally administering to the subject at least once daily (e.g., twice daily) at least one dosage form comprising an oily suspension comprising octreotide, wherein the octreotide in each dosage form is from about 18 mg to about 22 mg, wherein the total amount of octreotide administered per day is from about 36 to about 44 mg; and subsequent to the administration, evaluating an IGF-1 level (and/or a GH level) in a subject and comparing the level to a reference standard; wherein if the IGF-1 level (and/or the GH level) is above the reference standard, increasing the total amount of octreotide administered per day to from about 54 to about 66 mg; wherein the administering occurs at least 2 hours after a meal, or at least 1 hour before a meal.

Another such embodiment of the invention relates to a method of titrating a patient having acromegaly, the method comprising orally administering to the subject at least once daily (e.g., twice daily) at least one dosage form comprising an oily suspension comprising octreotide, wherein the octreotide in each dosage form is from about 18 mg to about 22 mg, wherein the total amount of octreotide administered per day is from about 54 to about 66 mg; and subsequent to the administration, evaluating an IGF-1 level (and/or a GH level) in a subject and comparing the level to a reference standard; wherein if the IGF-1 level (and/or the GH level) is above the reference standard, increasing the total amount of octreotide administered per day to from about 72 to about 88 mg; wherein the administering occurs at least 2 hours after a meal or at least 1 hour before a meal.

In one embodiment of the invention, if a capsule containing about 30 mg octreotide is administered, then the above algorithm is used to adjust the dose from 60 mg daily to 90 mg daily and a maximum of 120 mg daily; wherein the administering occurs at least 2 hours after a meal, or at least 1 hour before a meal. In another embodiment, if a capsule containing about 30 mg octreotide is administered, then the above algorithm is used to adjust the dose from 30 mg daily (only one capsule taken) to 60 mg daily (two capsules) to 90 mg daily (three capsules) and a maximum of 120 mg daily (four capsules); wherein the administering occurs at least 2 hours after a meal, or at least 1 hour before a meal.

In a further embodiment of the invention, if a capsule containing less than 20 mg octreotide is administered e.g., 10 mg, then the above algorithm is adjusted concomitantly.

In further embodiments of the titrating invention the oily suspension is formulated into a capsule; the capsule is enterically coated; the oral administration is twice daily comprising a first and second administration; the subject is dosed every 8-16 hours (e.g., every 12 hours); one administration takes place at least 6, 8, 10 or 12 hours before a second administration; and the subject is a human. In a further embodiment of the titrating invention the first administration prior to evaluation includes one or two dosage forms and the second administration includes one or two dosage forms. In a further embodiment of the titrating invention, the first daily administration prior to evaluation includes one dosage form and the second daily administration prior to evaluation includes one dosage form. In a further embodiment of the titrating invention the first daily administration prior to evaluation includes two dosage forms and the second daily administration prior to evaluation includes one dosage form. In a further embodiment of the titrating invention the first daily administration after evaluation includes two dosage forms and the second daily administration after evaluation includes two dosage forms. In a further embodiment of the invention one dosage form is administered once a day and two dosage forms are administered once a day, prior to evaluation. In a further embodiment of the invention two dosage forms are administered twice daily after evaluation. Administering occurs at least 2 hours after a meal, or at least 1 hour before a meal.

In a further embodiment of the invention each dosage form comprises from about 19 to about 21 mg of octreotide, more particularly 20 mg of octreotide which is about 3% w/w octreotide. In a further embodiment of the invention the total amount of octreotide administered per day prior to evaluation is from about 36 to about 44 mg (e.g., from about 38 to about 42 mg, or 40 mg). In a further embodiment of the invention the total amount of octreotide administered per day prior to evaluation is from about 54 to about 66 mg (e.g., from about 57 to about 63 mg, or 60 mg).

In a further embodiment of the invention the total amount of octreotide administered per day subsequent to evaluation is from about 54 to about 66 mg (e.g., from about 57 to about 63 mg, or 60 mg). In a further embodiment of the invention the total amount of octreotide administered per day subsequent to evaluation is from about 72 to about 88 mg (e.g., from about 76 to about 84 mg, or 80 mg). In a further embodiment of the invention the evaluation takes place at least two months from start of therapy (i.e., from start of administration of the dosage forms), 2-5 months from start of therapy or after 5 months from start of therapy (e.g., after 5, 6, 7 or 8 months or more from start of therapy).

In a specific embodiment of the invention the blood levels of IGF-1 and clinical symptoms are monitored when oral octreotide capsule dosage at 40 mg (20 mg BID), and if IGF-1 is normal and clinical symptoms are controlled or response level (biochemical and symptomatic response) is maintained, then oral octreotide capsule dosage is continued at 40 mg (e.g., 20 mg BID). In a further specific embodiment of the invention the blood levels of IGF-1 and clinical symptoms are further monitored when oral octreotide capsule dosage is at 40 mg, and if IGF-1 is not normal and clinical symptoms are not controlled or response level (biochemical and symptomatic response) is not maintained, then oral octreotide capsule dosage is increased to 60 mg daily (e.g., 40 mg morning+20 mg evening). In a further specific embodiment of the invention the blood levels of IGF-1 and clinical symptoms are further monitored when oral octreotide capsule dosage is at 60 mg, and if IGF-1 is normal and clinical symptoms are controlled or response level (biochemical and symptomatic response) is maintained, then oral octreotide capsule dosage is continued at 60 mg daily. In a further specific embodiment of the invention the blood levels of IGF-1 and clinical symptoms are further monitored when oral octreotide capsule dosage is at 60 mg, and if IGF-1 is not normal and clinical symptoms are not controlled or response level (biochemical and symptomatic response) is not maintained, then oral octreotide capsule dosage is increased to 80 mg (e.g., 40 mg morning+40 mg evening)

In a further embodiment of the invention the reference standard is an in vivo amount of growth hormone integrated over 2 hours is obtained which is equal or less than 2.5 ng/mL (for example equal or less than 1.0 ng/mL). In a further embodiment of the invention the reference standard is an in vivo concentration of IGF-I is obtained of equal or less than 1.3 times the upper limit of the age-adjusted normal range (ULN).

In a further embodiment of the invention an in vivo mean peak plasma concentration upon administration of octreotide after evaluation is about 3.5+/−0.5 ng/mL. In a further embodiment of the invention an in vivo mean area under the curve upon administration of octreotide after evaluation is about 15+/−4 h×ng/mL. In a further embodiment of the titrating invention the subject has had prior treatment for acromegaly which was surgical and/or pharmaceutical e.g., the pharmaceutical treatment was a somatostatin receptor ligand e.g., octreotide or lanreotide and was administered by injection. In a further embodiment of the titrating invention prior treatment of the subject with a somatostatin analog has been shown to be effective and tolerated. In a further embodiment of the invention the prior pharmaceutical treatment was pegvisomant or a dopamine agonist e.g., cabergoline.

In a further embodiment of the invention, prior treatment of the subject produced an IGF-1 level in the subject of equal or less than 1.0 to 1.5 times upper limit of normal (ULN) e.g., equal or less than 1.3 times upper limit of normal (ULN). In a further embodiment of the invention prior treatment of the subject produced 2-hour integrated growth hormone (GH) of less than 2.5 ng/mL e.g., less than 1.0 ng/mL.

A further embodiment of the invention is a method of predicting subsequent response to oral octreotide capsules in a patient receiving injectable treatment. Thus an embodiment of the invention is a method of predicting subsequent response to oral octreotide capsules comprising the oily suspension in a patient suffering from acromegaly, the method comprising measuring the degree of baseline control on injectable SRLs; and thereby determining if the patient is likely to respond to the oral octreotide capsules. In an embodiment of the invention the desired baseline control is IGF-I≤1ULN and GH<2.5 ng/mL when the patient is maintained on low to mid doses of injectable SRLs (octreotide <30 mg or lanreotide <120 mg).

In a particular embodiment the octreotide is formulated in assignee's proprietary formulation denoted as Transient Permeability Enhancer, TPE®. TPE is a combination of excipients assembled in a process leading to an oily suspension of hydrophilic particles containing medium-chain fatty acid salts and the active pharmaceutical ingredient (herein octreotide) suspended in a lipophilic medium and incorporated into a capsule. See co-assigned U.S. Pat. No. 8,535,695, which is incorporated by reference herein.

Dosages comprising octreotide should be not be administered with food. Dosages comprising octreotide should be administered preferably one hour before a meal or two hours after a meal i.e., on an empty stomach.

In some embodiments, a method of treating a patient suffering from acromegaly or suffering from severe diarrhea or flushing episodes associated with metastatic carcinoid tumor is disclosed comprising providing, selling or delivering any of the kits disclosed herein to a hospital, physician or patient.

The invention will be more fully understood by reference to the following example which details an exemplary embodiment of the invention. This example should not, however, be construed as limiting the scope of the invention. All citations throughout the disclosure are hereby expressly incorporated by reference.

While the present invention has been described in terms of various embodiments and examples, it is understood that variations and improvements will occur to those skilled in the art.

### EXAMPLE

Drug-Drug Interaction Study with Digoxin, Lisinopril and Effect of Ingested Water Volume on MYCAPSSA

### Study Description

This was a single-center, randomized, single-blind, placebo-controlled study in 18 healthy subjects. Two groups of subjects were studied in a 4-treatment, 2-sequence, and crossover design. The study was comprised of a screening period, a treatment period, and a follow-up period (approximately 10-13 days following the last test condition). Subjects were randomized in a 1:1 ratio to one of 2 sequences (i.e., Group 1 or 2) of 4 test conditions (i.e., A-D) as shown in Table 1.

TABLE 1
Test Conditions

| | Group 1 (n = 9) | Group 2 (n = 9) |
|---|---|---|
| Test | A | B |
| Conditions | B | A |
| | C | D |
| | D | C |

Test Conditions
A: MYCAPSSA 40 mg + 240 mL water
B: MYCAPSSA 40 mg + 75 mL water
C: MYCAPSSA 40 mg + 240 mL water + digoxin 0.5 mg + lisinopril 20 mg
D: placebo + 240 mL water + digoxin 0.5 mg + lisinopril 20 mg

### Methods

The PK of MYCAPSSA (40 mg octreotide (i.e., single dose of oral octreotide acetate 40 mg (2×20 mg capsules)) was assessed by analysis of data from Test Condition A. The effect of the volume of water ingested during drug administration on the absorption of MYCAPSSA was assessed by comparison of Test Conditions A and B. The effect of MYCAPSSA (40 mg) on the absorption of digoxin and lisinopril was assessed by comparison of Test Conditions C and D. Blood for PK analysis of octreotide and probe drugs was sampled serially following dosing as shown in Table 2. Blood sampling for digoxin and lisinopril was conducted over the first 12 hours after administration to assess the potential impact of the TPE formulation on the absorption profile.

TABLE 2
Timing of Pharmacokinetic Blood Samples

| | Test | |
|---|---|---|
| Analyte | Condition | Timing of PK blood samples |
| Octreotide | A and B | 0 (pre-dose, within 60 minutes before dosing), |
| | | every 30 minutes post-dose for the first 5 hours, |
| | | and then at 6 hr, 9 hr, 12 hr, and 24 hr post- |
| | | dose, ±5 min during each test condition |
| Octreotide | C and D | 1 hr, 3 hr, and 5 hr post-dose (±5 min) during |
| | | each test condition |
| Digoxin | C and D | 0 (pre-dose, up to 60 minutes before administra- |
| | | tion), 15 min, 30 min, 45 min, 1 hr, 1.25 hr, |
| | | 1.5 hr, 1.75 hr, 2 hr, 2.25 hr, 2.5 hr, 3 hr, 4 hr, |
| | | 5 hr, 6 hr, 8 hr, and 12 hr post-dose |
| Lisinopril | C and D | 0 (pre-dose, up to 60 minutes before administra- |
| | | tion), 30 min, 1 hr, 2 hr, 3 hr, 4 hr, 5 hr, 5.5 hr, |
| | | 6 hr, 6.5 hr, 7 hr, 7.5 hr, 8 hr, 8.5 hr, 9 hr, and |
| | | 12 hr post-dose (±5 min) |

Test Conditions
A: MYCAPSSA 40 mg + 240 mL water
B: MYCAPSSA 40 mg + 75 mL water
C: MYCAPSSA 40 mg + 240 mL water + digoxin 0.5 mg + lisinopril 20 mg
D: placebo + 240 mL water + digoxin 0.5 mg + lisinopril 20 mg
PK = pharmacokinetic

The PK parameters Cₘₐₓ, AUC₀₋ₜ, and AUC_{inf} for octreotide were compared between treatment conditions A and B using an ANOVA statistical model with treatment, period, sequence, and subject within sequence as the classification variables, using the natural logarithms of the data. Confidence intervals (CIs, (90%)) were constructed for the geometric mean rations (GMRs) (240 mL to 75 mL) of the 3 parameters using the natural log-transformed data and the 2 one-sided t-tests procedure. The GMRs and CIs were exponentiated back to the original scale. The effect of water volume was assessed from the GMRs and 90% CIs.

The discrete parameters Tₘₐₓ and T_{lag} were compared among treatments using non-parametric analyses.

Plasma concentrations of octreotide after administration of Test Condition C were used to confirm administration of MYCAPSSA but were not subjected to any statistical analyses.

A total of 18 healthy male and female subjects were enrolled into the study. Seventeen (17) subjects completed all 4 treatments and 1 subject, Subject 12, completed 2 of the 4 treatments—Test Conditions A and B.

Results

Effect of Water Volume on Octreotide Absorption

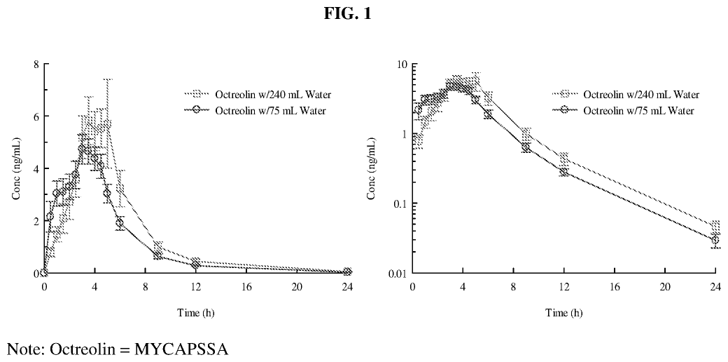

FIG. 1 displays mean (i standard error) plasma concentrations of octreotide after oral administration of a single 40-mg (2×20 mg octreotide) dose of MYCAPSSA with 240 mL and 75 mL of water to healthy volunteers under fasted conditions.

FIG. 1 and Table 3 show that administration of MYCAPSSA with 75 mL rather than 240 mL of water resulted in a slight decrease in the mean plasma octreotide concentrations. Examination of the graphs of the individual subject data showed a decrease in 7 of 18 individual subjects. The mean values for Cmax, AUC0-t, and AUCinf were decreased when MYCAPSSA was administered with 75 mL of water. The GMRs for the 3 parameters ranged from 79.01% to 88.78% and lower limits for the 90% CIs were <80.00% for all 3 parameters; the upper limit for Cmax was also >125.00% (see Table 4).

One (1) subject exhibited an apparent lag time (2 h) when MYCAPSSA was administered with 75 mL of water (Subject 01) and 1 subject had an apparent lag time (2 h) after administration with 240 mL of water (Subject 08).

The median and associated ranges for Tₘₐₓ were similar regardless of the volume of water and the mean t_{1/2} was comparable for both treatments (see Table 3).

The geometric mean AUCs shows a 20% decrease in exposure following administration of MYCAPSSA with 75 mL of water compared to administration with 240 mL of water. This decrease was variable among the subjects and was demonstrated in 9 subjects out of the 17 subjects, ranging from 7.52% to 78.4%. However, the remaining 8 subjects had increases ranging from 6.56% to 78.1%.

TABLE 3
Summary of Octreotide Pharmacokinetic Parameters
After Oral Administration of Single 40-mg (2 ×
20 mg) Doses of MYCAPSSA With 240 mL and 75 mL of
Water to Healthy Volunteers Under Fasted Conditions

| | MYCAPSSA with | MYCAPSSA with |
|---|---|---|
| Parameter* | 240 mL water | 75 mL water |
| T_{lag} (h)† | 2.00 (1) | 2.00 (1) |
| | [2.00-2.00] | [2.00-2.00] |
| Cₘₐₓ (ng/mL) | 8.21 ± 7.18 (18) | 6.13 ± 2.11 (18) |
| Tₘₐₓ (hr) | 3.54 (18) | 3.00 (18) |
| | [3.00-6.00] | [0.50-4.53] |
| AUC₍₀₋ₜ₎ (h × ng/mL) | 33.4 ± 20.9 (18) | 26.5 ± 11.1 (18) |
| AUC_{(inf)} (h × ng/mL) | 35.3 ± 20.4 (17) | 26.8 ± 11.1 (18) |
| λz (1/h) | 0.2120 ± 0.0736 (17) | 0.2496 ± 0.0843 (18) |
| t_{1/2} (h) | 3.54 ± 0.88 (17) | 3.05 ± 0.90 (18) |

*Mean ± standard deviation (N) except T_{lag} and Tₘₐₓ for which the median (N) [Range] is reported.
†A lag time was not calculated if the first concentration ≥ LOQ occurred at the first sample time.
AUC = area under the curve;
Cₘₐₓ = maximum plasma concentration;
λz = elimination rate constant;
LOQ = limit of quantitation;
t_{1/2} = elimination half-life;
T_{lag} = absorption lag time;
Tₘₐₓ = time of maximum plasma concentration

TABLE 4
Statistical Comparison of Octreotide Pharmacokinetic
Parameters After Oral Administration of Single 40-mg
(2 × 20 mg) Doses of MYCAPSSA With 240 mL and 75
mL of Water to Healthy Volunteers Under Fasted Conditions

| | Geometric Mean* | | Geometric Mean Ratio (%)^{†} | |
|---|---|---|---|---|
| Parameter | Test | Reference | Estimate | 90% Confidence Interval |

MYCAPSSA with 75 mL vs 240 mL Water

| | | | | |
|---|---|---|---|---|
| Cₘₐₓ | 5.82 | 6.55 | 88.78 | 62.68 → 125.74 |
| AUC₍₀₋ₜ₎ | 24.59 | 27.84 | 88.34 | 64.61 → 120.78 |
| AUC_{(inf)} | 24.92 | 31.54 | 79.01 | 61.41 → 101.64 |

*Least squares geometric means. Based on analysis of natural log-transformed pharmacokinetic parameters.
^{†}Lower confidence interval limits <80.00% and upper confidence interval limits >125.00% are shown in bold.
AUC = area under the curve;
Cₘₐₓ = maximum plasma concentration

Based on Cₘₐₓ and AUC₀₋ₜ, water volume appears to have no meaningful effect on systemic absorption of octreotide from MYCAPSSA (Table 5).

TABLE 5
Effect of Water Volume on Pharmacokinetic
Parameters of AUC₀₋ₜ and Cₘₐₓ

| | MYCAPSSA with | MYCAPSSA with |
|---|---|---|
| | 240 mL water | 75 mL water |
| Cₘₐₓ | 8.21 ± 7.18 (18) | 6.13 ± 2.11 (18) |
| AUC₀₋ₜ | 33.4 ± 20.9 (18) | 26.5 ± 11.1 (18) |

AUC₀₋ₜ = area under the concentration-time curve to the last quantifiable sample;
Cₘₐₓ = maximum plasma concentration

Effect of MYCAPSSA on Digoxin Absorption

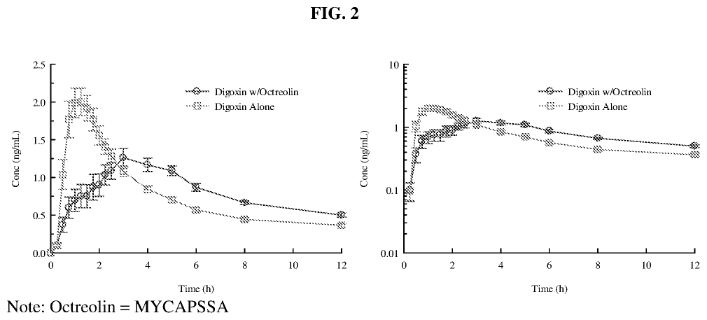

FIG. 2 displays mean (±standard error) plasma concentrations of digoxin after oral administration of single 0.5-mg doses to healthy volunteers with and without a single 40-mg (2×20 mg) dose of MYCAPSSA—Linear and Semi-logarithmic Axes.

Administration of digoxin concomitantly with MYCAPSSA resulted in no change to the extent but a decrease in the rate of digoxin absorption. This was evident from the mean (FIG. 2) and the majority (14) of the 17 individual subject plasma concentration-time graphs.

Although there was essentially no change in digoxin AUC₀₋₁₂ₕ (GMR 103.33%, 90% CI 94.43% to 113.08%), the arithmetic (Table 6) and geometric means (Table 7) for Cₘₐₓ were lower after concomitant administration, with a GMR of 62.87% and a 90% CI outside of the 80.00% to 125.00% window (Table 7).

The median Tₘₐₓ increased 3-fold from 1 to 3 hours (Table 6) during co-administration with MYCAPSSA. This range was, however, within the range of Tₘₐₓ indicated in the labeling for digoxin.

In summation, the lack of an apparent change in digoxin AUC, the decreased digoxin Cₘₐₓ, and the increased Tₘₐₓ are indicative of a decreased rate of absorption but no change in the extent of absorption of digoxin when co-administered with MYCAPSSA.

TABLE 6
Summary of Digoxin Pharmacokinetic Parameters
After Oral Administration of Single 0.5-mg Doses
to Healthy Volunteers With and Without a Single
40-mg (2 × 20 mg) Dose of MYCAPSSA

| Parameter* | Digoxin with MYCAPSSA | Digoxin alone |
|---|---|---|
| Cₘₐₓ (ng/mL) | 1.55 ± 0.46 (17) | 2.50 ± 0.71 (17) |
| Tₘₐₓ (h) | 3.00 (17) | 1.00 (17) |
| | [0.75-5.00] | [0.75-2.50] |
| AUC₍₀₋₁₂₎ (h × ng/mL) | 9.42 ± 2.09 (17) | 9.14 ± 2.03 (17) |

*Mean ± standard deviation (N) except T_{lag} and Tₘₐₓ for which the median (N) [Range] is reported.
†A lag time was not calculated if the first concentration ≥ LOQ occurred at the first sample time.
AUC = area under the curve;
Cₘₐₓ = maximum plasma concentration;
LOQ = limit of quantitation;
T_{lag} = absorption lag time;
Tₘₐₓ = time of maximum plasma concentration

TABLE 7
Statistical Comparison of Pharmacokinetic Parameters
for Digoxin After Oral Administration of Single
0.5-mg Doses to Healthy Volunteers With and Without
a Single 40-mg (2 × 20 mg) Dose of MYCAPSSA

| | | |
|---|---|---|
| Parameter | Geometric Mean* | Geometric Mean Ratio (%)^{†} |

Digoxin^{‡} with MYCAPSSA vs Alone

| | | | | |
|---|---|---|---|---|
| Cₘₐₓ | 1.50 | 2.38 | 62.87 | 55.10 → 71.75 |
| AUC₍₀₋₁₂₎ | 9.21 | 8.91 | 103.33 | 94.43 → 113.08 |

*Least squares geometric means based on analysis of natural log-transformed pharmacokinetic parameters.
^{†}Lower confidence interval limits <80.00% and upper confidence interval limits >125.00% are shown in bold.
^{‡}Lisinopril was co-administered with digoxin.
AUC = area under the curve;
Cₘₐₓ = maximum (peak) plasma concentration

Effect of MYCAPSSA on Lisinopril Absorption

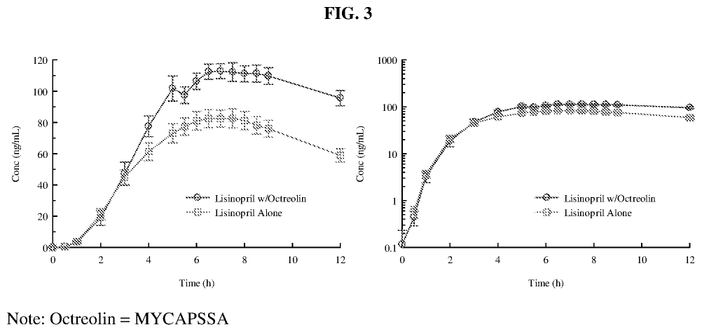

FIG. 3 displays the mean (±standard error) plasma concentrations of lisinopril after oral administration of single 20-mg doses to healthy volunteers with and without a single 40-mg (2×20 mg) dose of MYCAPSSA: linear and semi-logarithmic axes.

Administration of lisinopril concomitantly with MYCAPSSA resulted in an increase in the initial extent of lisinopril absorption. This was evident from the mean (Table 8) and the majority (11) of individual subject plasma concentration-time graphs. The arithmetic (Table 8) and geometric (Table 9) means for lisinopril Cₘₐₓ and AUC₀₋₁₂ₕ were higher after concomitant administration with GMRs of 150.33% and 139.74%, respectively, and 90% CIs outside of the 80.00% to 125.00% window (Table 9).

Of the 17 subjects completing both Test Conditions C and D, lisinopril AUC₀₋₁₂ decreased in 3 subjects, with decreases ranging from 6.17% to 16.1%. The remaining 14 subjects had increases in AUC₀₋₁₂ ranging from 2.98% to 140%. Of those 14 subjects with an increase in AUC₀₋₁₂, 11 subjects (79%) had an increase ≥25%.

The median and range for Tₘₐₓ were comparable when lisinopril was administered alone or with MYCAPSSA (Table 8), indicating no apparent change in the rate of absorption.

The initial extent of absorption of lisinopril is increased when taken concomitantly with MYCAPSSA.

TABLE 8
Summary of Lisinopril Pharmacokinetic Parameters After Oral
Administration of Single 20 mg Doses to Healthy Volunteers
With and Without a Single 40 mg (2 × 20 mg) Dose of MYCAPSSA

| | Lisinopril With | |
|---|---|---|
| Parameter* | Octreotide Capsules | Lisinopril Alone |
| Cₘₐₓ (ng/mL) | 128 ± 27.9 (17) | 87.0 ± 24.2 (17) |
| Tₘₐₓ (h) | 7.00 (17) | 7.50 (17) |
| | [5.00-9.00] | [6.50-9.00] |
| AUC₍₀₋₁₂₎ (h × ng/mL) | 939 ± 189 (17) | 690 ± 207 (17) |

*Mean ± standard deviation (N) except T_{lag} and Tₘₐₓ for which the median (N) [Range] is reported.
†A lag time was not calculated if the first concentration ≥ LOQ occurred at the first sample time.
AUC = area under the curve;
Cₘₐₓ = maximum plasma concentration;
LOQ = limit of quantitation;
T_{lag} = absorption lag time;
Tₘₐₓ = time of maximum plasma concentration

TABLE 9
Statistical Comparison of Pharmacokinetic Parameters
for Lisinopril After Oral Administration of Single
20 mg Doses to Healthy Volunteers With and Without
a Single 40 mg (2 × 20 mg) Dose of MYCAPSSA

| | Geometric Mean* | | Geometric Mean Ratio (%)^{†} | |
|---|---|---|---|---|
| Parameter | Test | Reference | Estimate | 90% Confidence Interval |

Lisinopril^{‡} with MYCAPSSA vs Alone

| | | | | |
|---|---|---|---|---|
| Cₘₐₓ | 125.65 | 83.58 | 150.33 | 132.00 → 171.20 |
| AUC₍₀₋₁₂₎ | 922.98 | 660.51 | 139.74 | 121.20 → 161.11 |

*Least squares geometric means. Based on analysis of natural log-transformed pharmacokinetic parameters.
^{†}Lower confidence interval limits <80.00% and upper confidence interval limits >125.00% are shown in bold.
^{‡}Digoxin was co-administered with lisinopril.
AUC = area under the curve;
Cₘₐₓ = maximum plasma concentration

### CONCLUSIONS

Administration of MYCAPSSA with 75 mL of water resulted in an approximate 20% decrease in octreotide exposure compared to administration with 240 mL of water. This decrease was variable among subjects; for the 17 subjects for whom AUC_{inf} could be calculated for both treatments, 9 subjects had decreases ranging from 7.52% to 78.4%. However, the remaining 8 subjects had increases ranging from 6.56% to 78.1%. Therefore, in general, water volume appears to have no meaningful effect on systemic absorption of octreotide from MYCAPSSA.

Administration of digoxin concomitantly with MYCAPSSA does not change the extent of digoxin absorption but decreases the rate of digoxin absorption; Cₘₐₓ decreased 37%, and Tₘₐₓ increased from 1 to 3 hours.

Administration of lisinopril concomitantly with MYCAPSSA increased the initial extent of absorption of lisinopril; Cₘₐₓ increased 50%, and AUC₀₋₁₂ increased 40%.

1.-7. (canceled)

8. A method of treating a subject in need of oral octreotide and lisinopril, the method comprising:
i) administering oral octreotide to a subject that is concomitantly administered lisinopril;
ii) monitoring the blood pressure of the subject; and
iii) if the blood pressure of the subject is reduced when the subject is co-administered oral octreotide relative to the blood pressure of the subject in the absence of oral octreotide, then adjusting the dose of lisinopril administered to the subject, wherein
the oral octreotide is administered in a dosage form, wherein the dosage form comprises:
a composition comprising a suspension which is an admixture of a hydr0phobic medium and a solid form, wherein the solid form comprises:
a therapeutically effective amount of octreotide and a medium chain fatty acid salt, and wherein the composition comprises 12% to 21% by weight of the medium chain fatty acid salt.

9. The method of claim 8, wherein the blood pressure of the subject is reduced relative to a reference standard.

10. The method of claim 9, wherein the reference standard is the blood pressure of the subject in the absence of oral octreotide.

11. The method of claim 9, wherein the reference standard is normal blood pressure which is less than 120/80.

12. The method of claim 8, wherein the method further comprises assessing the bioavailability of lisinopril in the subject.

13. The method of claim 12, wherein the bioavailability of the lisinopril is increased when the subject is co-administered oral octreotide relative to a reference standard.

14. The method of claim 13, wherein the reference standard is the blood pressure of the subject in the absence of oral octreotide.

15. The method of claim 13, wherein the reference standard is normal blood pressure which is less than 120/80.

16. The method of claim 8, wherein the composition comprises 12% to 18% by weight of the medium chain fatty acid salt.

17. The method of claim 8, wherein the medium chain fatty acid salt has a chain length from about 6 to about 14 carbon atoms.

18. The method of claim 8, wherein the medium chain fatty acid salt is sodium hexanoate, sodium heptanoate, sodium octanoate, sodium nonanoate, sodium decanoate, sodium undecanoate, sodium dodecanoate, sodium tridecanoate, or sodium tetradecanoate, or a corresponding potassium, lithium, or ammonium salt thereof, or a combination thereof.

19. The method of claim 8, wherein the medium chain fatty acid salt is sodium octanoate.

20. the method of claim 19, wherein the sodium octanoate is present in an amount of 12% to 18% by weight.

21. The method of claim 8, wherein the solid form further comprises a matrix forming polymer.

22. The method of claim 21, wherein the composition comprises 5% to 15% by weight of the matrix forming polymer.

23. The method of claim 21, wherein the matrix forming polymer is dextran or polyvinylpyrrolidone.

24. The method of claim 23, wherein the matrix forming polymer is polyvinylpyrrolidone.

25. The method of claim 24, wherein polyvinylpyrrolidone is present in the composition as an amount of about 5% to about 15% by weight.

26. The method of claim 24, wherein the medium chain fatty acid is sodium octanoate.

27. The method of claim 8, wherein the composition comprises 12% to 21% by weight of sodium octanoate, 5% to 10% by weight of polyvinylpyrrolidone, 20% to 80% by weight of glyceryl tricaprylate, and 3% to 10% by weight of a surfactant.

28. The method of claim 27, wherein the surfactant is glyceryl monocaprylate or polyoxyethylene sorbitan monooleate, or a combination thereof.

29. The method of claim 27, wherein the polyvinylpyrrolidone has a molecular weight of about 3000.

30. The method of claim 8, wherein the dosage form is an enterically coated capsule.
